(19) United States
(12) Patent Application Publication
(10) Pub. No.: US 2012/0220834 A1
(43) Pub. Date: Aug. 30, 2012

(54) STRAP BASED PHYSIOLOGICAL INFORMATION MONITORING SYSTEM HAVING AN ELECTRICAL CIRCUIT/S COVERING CASE WITH LOOP/S AND SENSOR BASES WITH LOOP/S

(76) Inventor: RAVINDRA WIJESIRIWARDANA, Bentonville, AR (US)

(21) Appl. No.: 13/037,244

(22) Filed: Feb. 28, 2011

(51) Int. Cl. A61B 5/00

(52) U.S. Cl. 600/301

(57) ABSTRACT

Present innovation describes a physiological information monitoring strap capable of monitoring at least one physiological information parameter such as ECG, heart rate, heart rate variability, skin temperature or respiration information. The system comprises of casing for the electronics with loop holes for the strap to be connected to the casing. Also the system is consists of sensor bases with loop holes so that the strap to be connected to the sensor bases. The electrical communications between the sensors of the sensor bases and the electrical circuits in the casing is done via wires or cables.

### CROSS-REFERENCE TO RELATED APPLICATIONS

This application claims the benefit of the USA non-provisional patent application 20090292193 filed 2009 Mar. 11 by the present inventor. This application claims the benefit of provisional patent application Ser. No. 61/035,852, filed 2008 Mar. 12 by the present inventor. This application claims the benefit of provisional patent application Ser. No. 61/302,320, filed 2010 Feb. 8 by the present inventor.

### FEDERALLY SPONSORED RESEARCH

Not Applicable

### SEQUENCE LISTING OR PROGRAM

Not Applicable

### BACKGROUND

1. Field

This application relates to bio-potential electrodes and sensors based wearable physiological information monitoring straps.

2. Prior Art and Operation

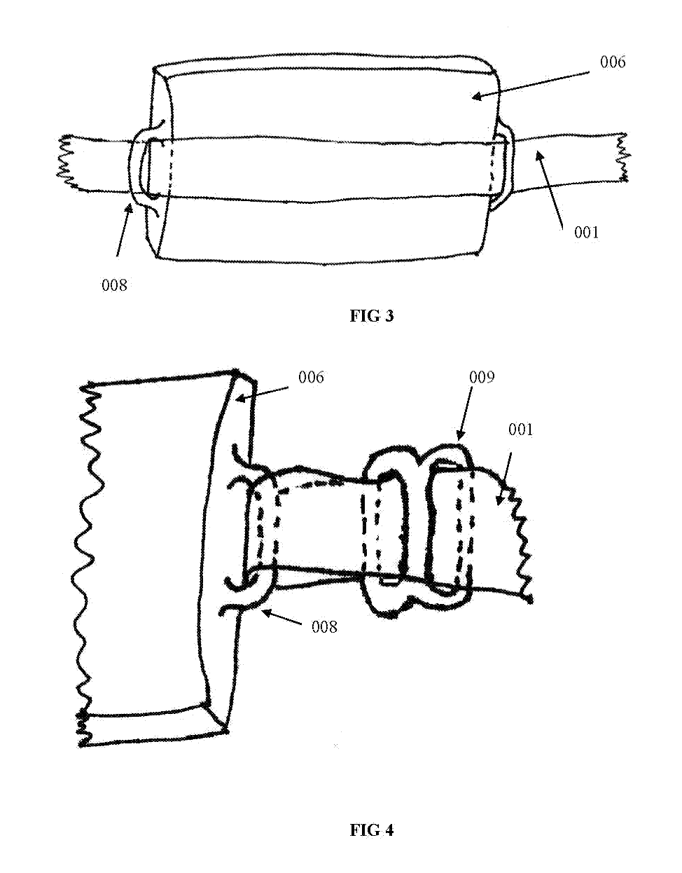

First part of the present innovation discloses an electrical covering case (006) having loop/s for the strap to be inserted and providing the physiological information monitoring unit to be securely connected to the strap. The covering case comprises of loop opening or loops openings (008) as shown in FIG 2. These openings are used to insert any portion of the strap (001) to secure to connect the casing to the strap (FIG. 3). These are smaller halls (011) in the body of the casing (006) for the electrical conductive wires (005) to carry the signal from the sensors to the electrical circuitry in side the case (FIG. 2)

The second part of the innovation is the sensor base (007) comprises of the loop hole/s (010). The strap (001) is inserted through these loop hole/s (010) be inserted so that the sensor base (007) is connected to the strap. The other side of the sensor base that on the skin side of a wearer comprises of the sensor element. This sensor element be a conductive bio-potential electrode or a bio potential electrode arrangement according to the innovation in U.S. patent application 61/035,852 filed by the present inventor in Mar. 12, 2008. Also this sensor base may contain temperature sensor/s or pressure sensor/s.

### DRAWINGS

### Figures

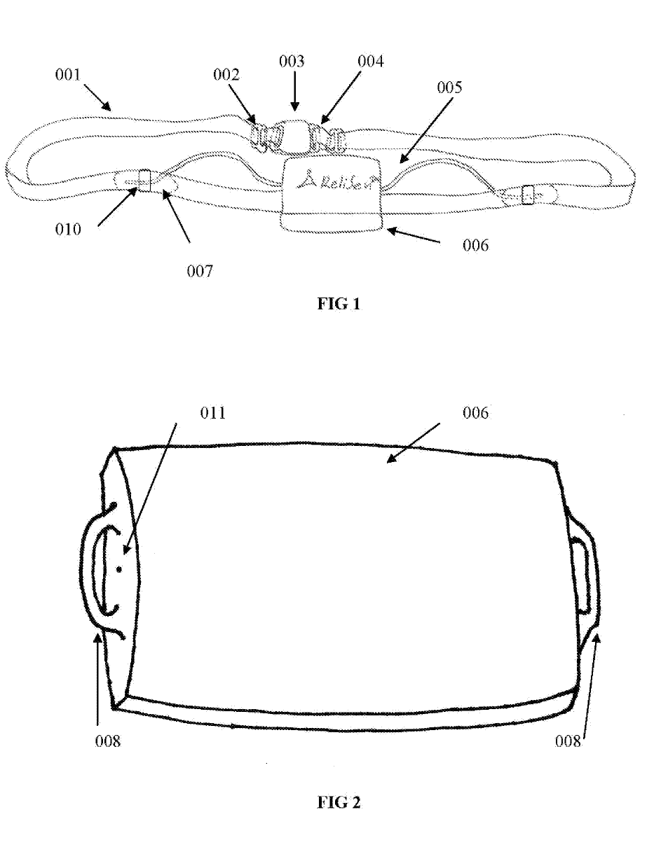

FIG. 1—shows the physiological information monitoring strap.

FIG. 2—shows the casing with loop holes

FIG. 3—shows the strap insert through the loop/s of the casing

FIG. 4—Shows the strap connected to a one side loop

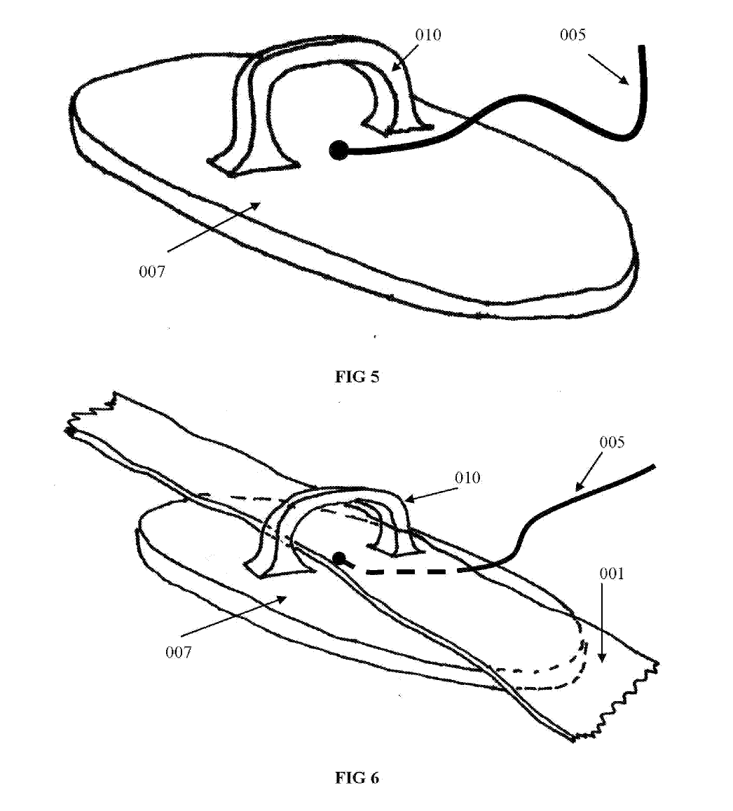

FIG. 5—Shows the sensor base with the loop

FIG. 6—Shows the strap insert through the loop hole of the sensor base

### REFERENCE NUMERALS

001—Strap

002—Strap connector arrangement double loop

003—Female part buckles part Strap connector arrangement

004—Male buckle part Strap connector arrangement

005—Electrode connector wire to the transmitter electronics

006—Transmitter electronics part containing casing with loops

007—Sensor/electrode base

008—Loops of the transmitter casing (006)

009—Strap to transmitter casing (006) connector arrangement double loop

010—Loop of the sensor base (007)

011—Holes of the casing (006) for the electrical wire (005)

I claim:

1. A chest wearable strap based physiological information monitoring device having a casing with at least one loop hole to insert any portion of the strap to connect the casing to the strap and this casing contains any part of the electrical circuitry needed for the physiological information monitoring.

2. A devices according claim 1 where the electrical circuit contains the signal processing, and wireless signal transmitting and receiving devices that communicate with an external base station.

3. A devise according to claim 2 where the electrical circuit/s contains an audio music and/or songs player and physiological information storage circuitry and signal processing circuitry.

4. A device according to claim 1 or claim 2 or claim 3 where the strap is inserted through at least two holes each on the opposite sides of the casing so that the strap is connected to the casing such that either the body of the casing is between the body of the wearer and the portion of the strap with the body of the casing or portion of the strap with the body of the casing is placed between the body of the wearer and the casing.

5. A device according to claim 1 or claim 2 or claim 3 where the portions of the strap is inserted through at least one hole of the casing and connected to the loop of the casing so that strap is not on the surface of the casing during wearing.

6. A sensor base having at least two opposite sides where at least one loop on one side and sensor element/s on the opposite side/s of the base so that the loop/s are used to insert a strap in order to connect the sensor base to a strap.

7. A sensor base according to claim 6 where the sensor element/s is a temperature sensor pressure sensor or an optical sensor or a bio potential electrode or any combination of.

8. A device according to claim 1 or claim 2 or claim 3 having at least one sensor base according to claim 6 of claim 7 and the sensors on the sensor base is connected to the electrical circuit inside the casing via wires or cables.

9. A device according to claim 8 where the physiological parameters being measured and monitored are individual or any combination of electro cardiogram, heart rate, heart rate variability respiration information and skin temperature.

10. A sensor base according to claim 6 where the materials used are plastic, elastic or any combination of the two.

11. A device according to claim 1 or claim 2 or claim 3 having at least one sensor on the strap or in the strap or embedded in the strap is connected to the electrical circuit inside the casing via wires or cables.

12. A device according to claim 11 where the sensor element/s is a temperature sensor pressure sensor or an optical sensor or a bio potential electrode or any combination of.

13. A device according to claim 12 where the physiological parameters being measured and monitored are individual or any combination of electro cardiogram, heart rate, heart rate variability respiration information and skin temperature.

14. A device according to claim 8 or claim 9 or claim 12 or claim 13 where the strap material is transparent, translucent or any color.

15. A device according to claim 8 or claim 9 or claim 12 or claim 13 where the strap material is acrylic, polyurethane or silicone or any combination of.
